(19) United States
(12) Patent Application Publication
(10) Pub. No.: US 2024/0153094 A1
(43) Pub. Date: May 9, 2024

(54) SYSTEMS AND METHODS FOR ANNOTATING TUBULAR STRUCTURES

(71) Applicant: Shanghai United Imaging Intelligence Co., Ltd., Shanghai (CN)

(72) Inventors: Yikang Liu, Cambridge, MA (US); Shanhui Sun, Lexington, MA (US); Terrence Chen, Lexington, MA (US)

(73) Assignee: Shanghai United Imaging Intelligence Co., Ltd., Shanghai (CN)

(21) Appl. No.: 17/981,988

(22) Filed: Nov. 7, 2022

(51) Int. Cl. G06T 7/11; G06T 7/00; G16H 50/50

(52) U.S. Cl. CPC G06T 7/11; G06T 7/0012; G16H 50/50; G06T 2207/30101

(57) ABSTRACT

Described herein are systems, methods, and instrumentalities associated with automatically annotating a tubular structure (e.g., such as a blood vessel, a catheter, etc.) in medical images. The automatic annotation may be accomplished using a machine-learning image annotation model and based on a marking of the tubular structure created or confirmed by a user. A user interface may be provided for a user to create, modify, and/or confirm the marking, and the ML model may be trained using a training dataset that comprises marked images of the tubular structure paired with ground truth annotations of the tubular structure.

### BACKGROUND

Having annotated data is crucial to the training of machine-learning (ML) models or artificial neural networks. Current data annotation relies heavily on manual work by qualified annotators (e.g., professional radiologists if the data includes medical images), and even when computer-based tools are provided, they still require a tremendous amount of human effort. This is especially true for annotating tubular structures, such as blood vessels, catheters, wires, etc., since these structures may be inherently thin and have irregular shapes. Accordingly, it is highly desirable to develop systems and methods to automate the image annotation process (e.g., for tubular structures and/or other organs or tissues of a human body) such that more data may be obtained for ML training and/or verification.

### SUMMARY

Described herein are systems, methods, and instrumentalities associated with automatic image annotation. According to one or more embodiments of the present disclosure, an apparatus configured to perform the automatic image annotation task may comprise at least one processor that is configured to provide a visual representation of a medical image comprising a tubular structure, and obtain, based on one or more user inputs, a marking of the tubular structure in the medical image. Based on the marking of the tubular structure and a machine-learned (ML) image annotation model, the processor may be further configured to generate, automatically, an annotation of the tubular structure such as a segmentation mask associated with the tubular structure, which may be stored or exported for various application purposes.

In examples, the tubular structure described herein may be an anatomical structure of a human body such as a blood vessel, or a medical device inserted or implemented into the human body such as a catheter or a guide wire. In examples, the marking of the tubular structure may include one or more lines drawn through or around the tubular structure that may be created using one or more sketch or annotation tools provided by the apparatus. At least one of these sketch or annotation tools may have a pixel-level accuracy, and the user inputs described herein may be received as a result of a user using the sketch or annotation tools. In examples, the apparatus describe herein may be further configured to generate, automatically, a preliminary marking of the tubular structure, and present the preliminary marking to a user of the apparatus, where the one or more user inputs may include actions that modify the preliminary marking.

In examples, the automatic annotation and/or the preliminary marking may be obtained using respective artificial neural networks (ANNs). For instance, the ML image annotation model may be implemented and learned using an ANN and based on a training dataset that may comprise marked images of the tubular structure paired with ground truth annotations (e.g., segmentation masks) of the tubular structure. During training, the ANN may be configured to predict a segmentation mask for the tubular structure based on a marked training image of the tubular structure and adjust parameters of the ANN based on a difference between the predicted segmentation mask and a corresponding ground truth segmentation mask.

### BRIEF DESCRIPTION OF THE DRAWINGS

A more detailed understanding of the examples disclosed herein may be had from the following description, given by way of example in conjunction with the accompanying drawing.

FIG. 1 is a diagram illustrating an example of automatically annotating a medical image comprising a tubular structure, in accordance with one or more embodiments of the present disclosure.

FIG. 2 is a diagram illustrating example techniques for automatically annotating a second 3D image dataset based on an annotated first 3D image dataset, in accordance with one or more embodiments of the present disclosure.

FIG. 3 is a flow diagram illustrating example operations that may be associated with training a neural network, in accordance with one or more embodiments of the present disclosure.

FIG. 4 is a block diagram illustrating example components of an apparatus that may be configured to perform the tasks described in accordance with one or more embodiments of the present disclosure.

### DETAILED DESCRIPTION

The present disclosure is illustrated by way of example, and not by way of limitation, in the figures of the accompanying drawings.

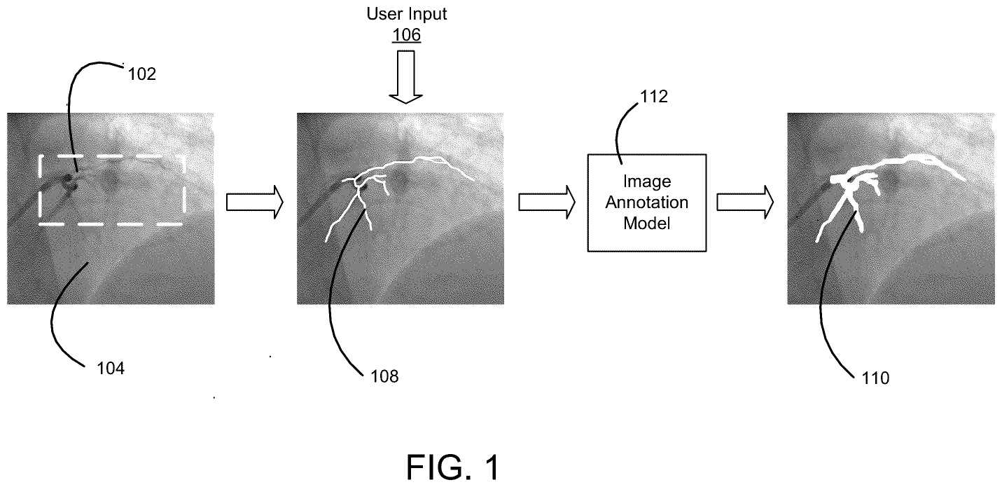

FIG. 1 illustrates an example of automatically annotating a tubular structure 102 in a medical image 104 in accordance with one or more embodiments of the present disclosure. The medical image 104 may be described in the example as a magnetic resonance imaging (MRI) image, but those skilled in the art will appreciate that the disclosed techniques may also be used to annotate other types of medical images (e.g., computed tomography (CT) images, X-ray images, etc.) or non-medical images that include a tubular structure. The tubular structure 102 (e.g., a blood vessel inside the dotted rectangular area) may be described as a blood vessel in the example, but those skilled in the art will appreciate that the disclosed techniques may also be applicable to other types of tubular structures (e.g., catheters, wires, or other types of medical devices inserted or implemented into a human body). An apparatus configured to perform the automatic annotation task illustrated in FIG. 1 may obtain the medical image 104 (e.g., from an MRI scanner or a medical image database), and provide a visual representation of the medical image 104 on a display device (e.g., which may or may not be part of the apparatus). The apparatus may do so, for example, by generating a graphical user interface (GUI) (not shown), and display the medical image 104 in an area of the GUI. The GUI may also include other areas or components such as, e.g., tools (e.g., brushes, erasers, etc.) for a user to create and/or modify (e.g., with a pixel-level granularity or accuracy) a marking 108 (e.g., a sketch) of the tubular structure 102. The marking 108 may include, for example, one or more lines drawn through or around the tubular structure 102 in the medical image 104, a bounding shape (e.g., a bound box) around the tubular structure 102, one or more marks made inside or around the tubular structure 102, etc. The tools described above may be activated and/or used, for example, with an input device such as a computer mouse, a computer keyboard, a stylus, or a tap or dragging motion of a user's finger on the display device (e.g., if the display device includes a touch screen), and one or more user inputs 106 may be received by the apparatus described herein as a result of activating and/or using the tools. In some examples, the one or more user inputs may create the marking 108 of the tubular structure 102, while in other examples, the apparatus described herein may automatically generate and present (e.g., via the same GUI described above) a preliminary marking (e.g., a preliminary sketch) of the tubular structure 102 (e.g., based on machine-learned (ML) segmentation model), and the one or more user inputs may confirm or modify (e.g., with a pixel-level granularity or accuracy) the preliminary marking so as to derive the marking 108 shown in FIG. 1.

When referred to herein, a marking of the tubular structure may include one or more lines drawn through or around the tubular structure 102, a rough outline of tubular structure 102, a bounding shape (e.g., a bounding box) around the tubular structure 102, etc. that may indicate the location, length, width, turning directions, branching directions, etc. of the tubular structure 102 in the medical image 104. The marking 108 may occupancy a plurality of pixels of the medical 104 that correspond to the tubular structure 102, but may not cover all the pixels of the tubular structure 102 (e.g., the marking 108 may roughly trace the tubular structure 102, but may not be accurate enough to serve as an annotation of the tubular structure 102).

Based on the marking 108, an annotation 110 (e.g., with a pixel-level accuracy) may be automatically generated using an ML image annotation model 112, as shown in FIG. 1. The annotation 110 may include, for example, a segmentation mask (e.g., a binary image having the same size as medical image 104 and comprising pixel values of zeroes and ones, where a value of zero may indicate that a corresponding pixel in the medical image 104 does not belong to the tubular structure 102 and a value of one may indicate that a corresponding pixel in the medical image 104 belongs to the tubular structure 102). The annotation 110 may cover all (e.g., substantially all) of the region of the medical image 104 that corresponds to the tubular structure 102 and, once generated, the annotation 110 may be stored and/or exported (e.g., to a local storage device such as a hard drive or a cloud-based storage device) for various purposes including, for example, training machine-learning models and facilitating clinical applications such as vessel reconstruction, vessel stenosis modeling, and catheter and wire localization (e.g., offline or during a surgical procedure). Hence, using the example techniques illustrated by FIG. 1, fast annotation of images containing tubular structures may be accomplished with minimum and easy-to-accomplish user inputs (e.g., the marking 108). The techniques may not only reduce costs associated with manual annotation, but also improve the outcome of operations or procedures that use the annotated images (e.g., since the automatically generated annotation may have pixel-level accuracy).

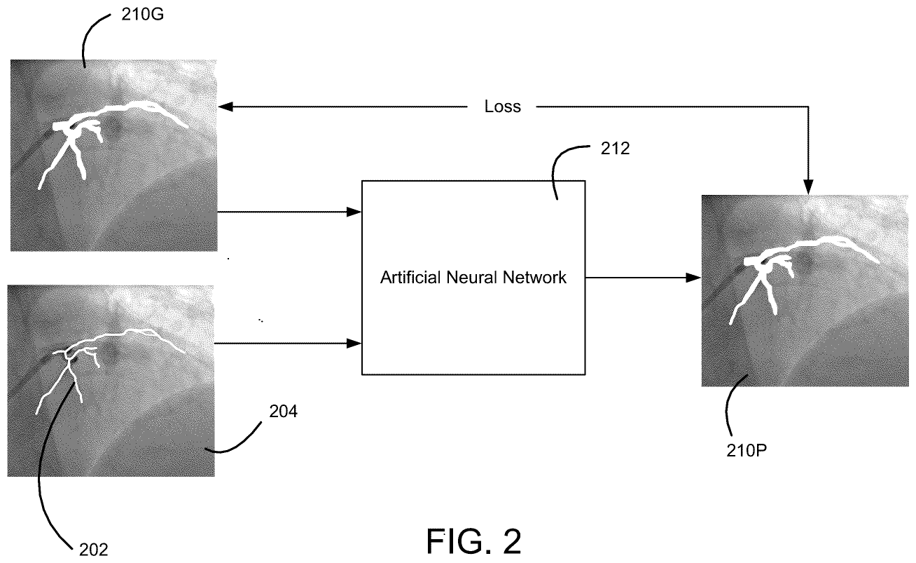

The ML image annotation model 112 may be implemented and/or learned using an artificial neural network (ANN), and based on a training dataset that comprises marked images of the tubular structure 102 (e.g., with sketches of the tubular structure) paired with ground truth annotations (e.g., segmentation masks) of the tubular structure. FIG. 2 illustrates an example of training an ANN 212 to learn the ML image annotation model 112 and perform the automatic annotation tasks described herein (the terms “machine-learning,” “machine-learned,” “artificial intelligence,” or “neural network” may be used interchangeably in describing the example of FIG. 2 and/or the other examples provided in the present disclosure). The ANN 212 may include a convolutional neural network (CNN) comprising an input layer and one or more convolutional layers, pooling layers, and/or fully-connected layers. The input layer may be configured to receive a two-channel tensor, where one channel may be associated with a training image (e.g., 204) and the other channel may be associated with a corresponding marked image (e.g., a sketch mask where pixels inside the sketch are given values of ones and pixels outside the sketch are given values of zeros) associated with a tubular structure (e.g., 202), and each of the convolutional layers may include a plurality of convolution kernels or filters with respective weights for extracting features associated with an object of interest from the image (e.g., the tubular structure 202) and the sketch (e.g., such as image gradient, image texture, the distance of an image pixel to a corresponding sketch mark), the respective topologies of the object of interest and the sketch, the topological similarity between the object of interest and the sketch, etc. The convolutional layers may be followed by batch normalization and/or linear or non-linear activation (e.g., such as a rectified linear unit (ReLU) activation function), and the features extracted through the convolution operations may be down-sampled through one or more pooling layers to obtain a representation of the features, for example, in the form of a feature vector or a feature map. The ANN 212 may further include one or more un-pooling layers and one or more transposed convolutional layers. Through the un-pooling layers, the features extracted through the operations described above may be up-sampled, and the up-sampled features may be further processed through the one or more transposed convolutional layers (e.g., via a plurality of deconvolution operations) to derive an up-scaled or dense feature map or feature vector. The dense feature map or vector may then be used to predict additional areas (e.g., at a pixel-level) of the medical image 204 that may correspond to the tubular structure of interest (e.g., by finding areas that contain the same or similar features as the marked areas).

The prediction 210P (e.g., in the form of a segmentation mask) made by the ANN 212 may be compared to a ground truth annotation (e.g., a ground truth segmentation mask) that may be paired with the marked medical image 204 in the training dataset. A loss associated with the prediction may be determined based on the comparison, for example, using a loss function such as a mean squared error (MSE), L1 norm, or L2 norm based loss function. The loss may be used to update the weights of the ANN 212 (e.g., parameters of the ML image annotation model), e.g., by backpropagating a gradient descent of the loss function through the ANN 212.

In examples, a neural network (e.g., a CNN) having a structure similar to that of ANN 212 may be used to generate, automatically, a preliminary marking or annotation of the tubular structure described herein, which may be presented to a user for modification and/or confirmation. The modified or confirmed marking or annotation may then be used as a basis to complete the automatic annotation task described here. The neural network may be, for example, an image segmentation neural network (e.g., an ML segmentation model) trained for segmenting a tubular structure from an input image. Since the segmentation (or marking) produced by such a neural network may be further refined by the ML image annotation model described herein, the training criteria (e.g., quality of the training data, number of training iterations, etc.) for the neural network may be relaxed and it may be sufficient for the neural network to only produce a coarse segmentation or marking of the tubular structure.

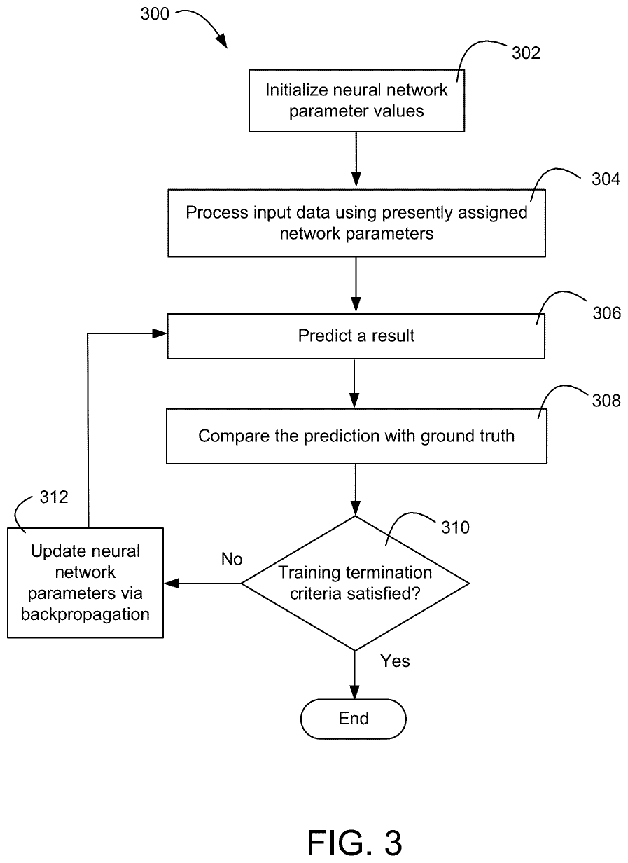

FIG. 3 illustrates example operations 300 that may be associated with training a neural network (e.g., a neural network used to implement the ML annotation or ML segmentation model described herein) to perform one or more of the tasks described herein. As shown, the training operations 300 may include initializing the operating parameters of the neural network (e.g., weights associated with various layers of the neural network) at 302, for example, by sampling from a probability distribution or by copying the parameters of another neural network having a similar structure. The training operations 300 may further include processing an input (e.g., a training image) using presently assigned parameters of the neural network at 304, and making a prediction for a desired result (e.g., an estimated annotation) at 306. The prediction result may be compared to a ground truth at 308 to determine a loss associated with the prediction, for example, based on a loss function such as mean squared errors between the prediction result and the ground truth, an L1 norm, an L2 norm, etc. At 310, the loss may be used to determine whether one or more training termination criteria are satisfied. For example, the training termination criteria may be determined to be satisfied if the loss is below a threshold value or if the change in the loss between two training iterations falls below a threshold value. If the determination at 310 is that the termination criteria are satisfied, the training may end; otherwise, the presently assigned network parameters may be adjusted at 312, for example, by backpropagating a gradient descent of the loss function through the network before the training returns to 306.

For simplicity of explanation, the training operations are depicted and described herein with a specific order. It should be appreciated, however, that the training operations may occur in various orders, concurrently, and/or with other operations not presented or described herein. Furthermore, it should be noted that not all operations that may be included in the training method are depicted and described herein, and not all illustrated operations are required to be performed.

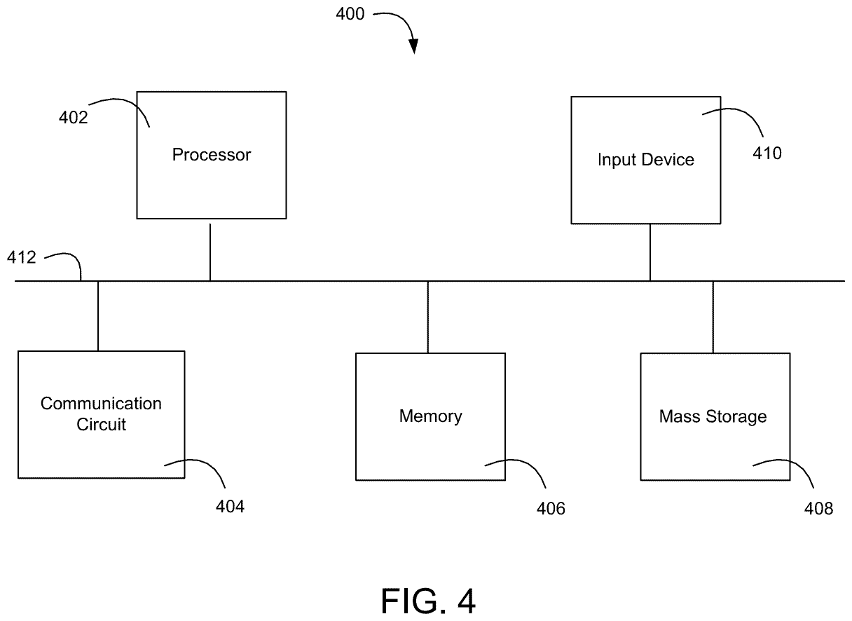

The systems, methods, and/or instrumentalities described herein may be implemented using one or more processors, one or more storage devices, and/or other suitable accessory devices such as display devices, communication devices, input/output devices, etc. FIG. 4 illustrates an example apparatus 400 that may be configured to perform the automatic image annotation tasks described herein. As shown, apparatus 400 may include a processor (e.g., one or more processors) 402, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 400 may further include a communication circuit 404, a memory 406, a mass storage device 408, an input device 410, and/or a communication link 412 (e.g., a communication bus) over which the one or more components shown in the figure may exchange information.

Communication circuit 404 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 406 may include a storage medium (e.g., a non-transitory storage medium) configured to store machine-readable instructions that, when executed, cause processor 402 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 408 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 402. Input device 410 may include a keyboard, a mouse, a voice-controlled input device, a touch sensitive input device (e.g., a touch screen), and/or the like for receiving user inputs to apparatus 400.

It should be noted that apparatus 400 may operate as a standalone device or may be connected (e.g., networked, or clustered) with other computation devices to perform the functions described herein. And even though only one instance of each component is shown in FIG. 4, a skilled person in the art will understand that apparatus 400 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. An apparatus, comprising:
at least one processor configured to:
provide a visual representation of a medical image, wherein the medical image includes a tubular structure associated with a human body;
obtain, based on one or more user inputs, a marking of the tubular structure in the medical image; and
generate, based on the marking of the tubular structure and a machine-learned (ML) image annotation model, an annotation of the tubular structure.

2. The apparatus of claim 1, wherein the annotation includes a segmentation mask associated with the tubular structure.

3. The apparatus of claim 1, wherein the marking of the tubular structure includes one or more lines drawn through or around the tubular structure.

4. The apparatus of claim 1, wherein the at least one processor being configured to obtain the marking of the tubular structure comprises the at least one processor being configured to:
generate, automatically, a preliminary marking of the tubular structure;
present the preliminary marking to a user of the apparatus; and
obtain the marking of the tubular structure based on the one or more user inputs that modify the automatically generated preliminary marking of the tubular structure.

5. The apparatus of claim 4, wherein the preliminary marking of the tubular structure is generated based on an ML image segmentation model.

6. The apparatus of claim 1, wherein the ML image annotation model is learned from a training dataset that comprises marked images of the tubular structure paired with ground truth annotations of the tubular structure.

7. The apparatus of claim 6, wherein the ML image annotation model is learned using an artificial neural network (ANN) and wherein, during training of the ANN, the ANN is configured to predict a segmentation mask for the tubular structure based on a marked training image of the tubular structure and adjust parameters of the ANN based on a difference between the predicted segmentation mask and a corresponding ground truth segmentation mask.

8. The apparatus of claim 1, wherein the at least one processor is further configured to provide one or more annotation tools to a user of the apparatus, and wherein the one or more user inputs are received as a result of the user using the one or more annotation tools.

9. The apparatus of claim 8, wherein at least one of the one or more annotation tools has a pixel-level accuracy.

10. The apparatus of claim 1, wherein the tubular structure includes a blood vessel of the human body or a medical device inserted or implemented into the human body.

11. The apparatus of claim 1, wherein the at least one processor is further configured to store or export the annotation of the tubular structure.

12. A method of image annotation, comprising:
providing a visual representation of a medical image, wherein the medical image includes a tubular structure associated with a human body;
obtaining, based on one or more user inputs, a marking of the tubular structure in the medical image; and
generating, based on the marking of the tubular structure and a machine-learned (ML) image annotation model, an annotation of the tubular structure.

13. The method of claim 12, wherein the annotation includes a segmentation mask associated with the tubular structure.

14. The method of claim 12, wherein the marking of the tubular structure includes one or more lines drawn through or around the tubular structure in the medical image.

15. The method of claim 12, wherein obtaining the marking of the tubular structure comprises:
generating, automatically, a preliminary marking of the tubular structure;
presenting the preliminary marking to a user; and
obtaining the marking of the tubular structure based on the one or more user inputs that modify the automatically generated preliminary marking of the tubular structure.

16. The method of claim 15, wherein the preliminary marking of the tubular structure is generated based on an ML image segmentation model.

17. The method of claim 12, wherein the ML image annotation model is learned from a training dataset that comprises marked images of the tubular structure paired with ground truth annotations of the tubular structure.

18. The method of claim 17, wherein the ML image annotation model is learned using an artificial neural network (ANN) and wherein, during training of the ANN, the ANN is configured to predict a segmentation mask for the tubular structure based on a marked training image of the tubular structure and adjust parameters of the ANN based on a difference between the predicted segmentation mask and a corresponding ground truth segmentation mask.

19. The method of claim 12, further comprising providing one or more annotation tools to a user, and wherein the one or more user inputs are received as a result of the user using the one or more annotation tools.

20. The method of claim 12, wherein the tubular structure includes a blood vessel of the human body or a medical device inserted or implemented into the human body.
